(19) United States
(12) Patent Application Publication
(10) Pub. No.: US 2023/0157801 A1
(43) Pub. Date: May 25, 2023

(54) FUNCTIONALIZED SYNTHETIC SURGICAL MESH

(71) Applicants: BAXTER INTERNATIONAL INC., Deerfield, IL (US); BAXTER HEALTHCARE SA, Glattpark(Opfikon) (CH)

(72) Inventors: Kevin Michael Lewis, Grayslake, IL (US); Mark Alan Nordhaus, Antioch, IL (US); John-Bruce D. Green, Buffalo Grove, IL (US); Vadim V. Krongauz, Bartlett, IL (US)

(21) Appl. No.: 17/988,352

(22) Filed: Nov. 16, 2022

Related U.S. Application Data
(60) Provisional application No. 63/282,570, filed on Nov. 23, 2021.

(51) Int. Cl. A61F 2/00

(52) U.S. Cl. CPC A61F 2/0063; A61F 2002/0068; A61F 2210/0004

(57) ABSTRACT

Disclosed herein are surgical mesh materials and methods of production and use thereof.

### CROSS-REFERENCE TO RELATED APPLICATION

The present application claims the benefit of U.S. Provisional Application No. 63/282,570, filed Nov. 23, 2021, the entire contents of which is incorporated by reference herein.

### FIELD

The present Specification relates to the production and use of surgical mesh materials.

### BACKGROUND

Surgical mesh is a medical device that supports damaged tissue, such as around a hernia, as it heals. Surgeons place the mesh across the area surrounding the hernia, attaching it with stitches, staples or glue. Pores in the mesh allow tissue to grow into the device. Surgical mesh is used in nine out of ten hernia surgeries annually in the U.S.

Several forms of surgical mesh are currently in use; patches are designed to go over or under the weakened or damaged tissue; plugs fit inside a hole in the tissue; and sheets can be custom cut and fitted for the patient's specific condition.

While effective, current mesh designs do not provide optimum performance, particularly in terms of cell in-growth and tissue adherence. Therefore, improved systems, devices, and methods are desirable.

### SUMMARY

The instant disclosure provides a novel class of fully synthetic biodegradable surgical meshes. Disclosed embodiments promote cell in-growth on the superficial mesh surface in contact with tissue in a competitive manner as compared to current biologic meshes, and display minimal tissue adherence on the surface in contact with the viscera.

Disclosed embodiments employ surface charges and specific charge patterning methods to create a scaffold with an architecture engineered for cell in-growth and the ability to provide the stability and strength needed for hernia meshes, as well as for controlled biodegradation.

Disclosed embodiments comprise scaffold surface coatings, for example comprising surface species immobilized by, for example, chemical or physical bonding. In embodiments, the surface species can comprise antimicrobials.

Disclosed embodiments also comprise methods of making the disclosed surgical mesh materials.

Disclosed embodiments also comprise methods of use of the disclosed surgical mesh materials.

Disclosed embodiments also comprise kits comprising the disclosed surgical mesh materials.

### DETAILED DESCRIPTION

Disclosed surgical mesh embodiments comprise synthetic surgical meshes with improved cell in-growth potential, minimal visceral tissue adherence, and biodegradation, thus improving patient outcomes. As fully synthetic devices, disclosed embodiments are distinguished by manufacturing processes and functionalization. For example, while current synthetic meshes are manufactured using a weave pattern of polymeric material(s), disclosed embodiments can comprise a uniform, non-woven polymeric material. Further, while current synthetic meshes employ multiple materials for specific functions, the instant disclosure provides polymeric materials functionalized with synthetic moieties to achieve specific treatment goals.

### Definitions:

“Administration,” or “to administer” means the step of giving (i.e. administering) a medical device, material or agent to a subject. The materials disclosed herein can be administered via a number of appropriate routes, but are typically employed in connection with a surgical procedure.

“Patient” means a human or non-human subject receiving medical or veterinary care.

“Therapeutically effective amount” means the level, amount or concentration of an agent, material, or composition needed to achieve a treatment goal.

“Treat,” “treating,” or “treatment” means an alleviation or a reduction (which includes some reduction, a significant reduction, a near total reduction, and a total reduction), resolution or prevention (temporarily or permanently) of a symptom, disease, disorder or condition, so as to achieve a desired therapeutic or cosmetic result, such as by healing of injured or damaged tissue, or by altering, changing, enhancing, improving, ameliorating and/or beautifying an existing or perceived disease, disorder or condition.

The instant disclosure provides synthetic surgical mesh materials comprising a biodegradable, synthetic mesh comprising a scaffold. In embodiments, the mesh is chemically functionalized to enhance wound healing, self-adherence, anti-adhesiveness, and bactericidal and/or bacteriostatic properties. In embodiments, the surface or surfaces of the mesh can be functionalized utilizing the inherent properties of the polymeric material(s) and/or through bound synthetic moieties. In embodiments the mesh can comprise one or more different polymeric laminations and/or weaves that are biodegradable.

In embodiments, the surgical mesh is charged to induce cellular in-growth.

In embodiments, the surgical mesh comprises at least one functionalized moiety to reduce visceral tissue adhesion.

In embodiments, the surgical mesh comprises an elutable antimicrobial as part of the anti-adhesive layer.

In embodiments, the surgical mesh is biodegradable.

### Surgical Mesh Scaffolding

The scaffolding of disclosed surgical mesh embodiments has two primary functions; 1) to provide mechanical integrity to the treatment area, such as the hernia, while the wound heals and 2) to provide a porous space within which superficial tissue cells can proliferate, thereby healing the wound.

The mechanical integrity of the scaffolding is determined by both the morphology and the mechanical properties of the polymers used to create the scaffold. Thus, disclosed embodiments comprise determination and production of scaffolding material suitable for a specific treatment goal.

In embodiments, scaffold morphology may comprise a porous, solid foam matrix, a woven nanofiber mesh, a patterned film, a hydrogel, or any combination thereof.

A secondary function of the scaffolding is to degrade as the wound is repaired. In embodiments, the synthetic, biodegradable polymeric material can comprise polypropylene (PP), polyethylene terephthalate (PET), expanded polytetrafluoroethylene (ePTFE), polycaprolactone (PCL), poly(L-lactide) (PLL), polyglycolic acid (PGA) and copolymers thereof, such as poly(lactic-coglycolic acid) (PLGA), poly(glycolide-co-caprolactone), and polyglycolide-co-trimethylene carbonate), etc.

The elastic profile and structural arrangement of the scaffolding are known to play crucial roles in cell intrusion. While several naturally occurring and woven structural motifs are commonplace in the industry, disclosed embodiments comprise engineering the structural arrangement in concert with appropriate polymeric materials to provide the proper environment for cell intrusion and subsequent tissue vascularization. For example, the mesh material that is chosen for accelerated in-growth and vascularization can be incompatible with the mechanical stresses encountered from hernia indications. Thus, in embodiments, disclosed scaffold materials can comprise multiple materials. For example, in an embodiment, the scaffold comprises a separate structural construct which has the strength to maintain adequate stability, and is made of polymers that will not rapidly degrade. In embodiments the multiple scaffold materials can be created simultaneously with the in-growth structures, or they can be made separately and combined by lamination or other methods.

Methods of producing the disclosed scaffolding materials can comprise for example, 3D printing, multi-inkjet printing, holographic printing, casting, embossing, photolithography or flexigraphic printing.

### Scaffold Surface Functionalization

In embodiments, surface functionalization of the polymeric mesh material(s) enhances cellular interactions, inhibits cellular interactions, increases adherence to superficial tissue surfaces, prevents tissue adherence to the viscera, and/or stops or reduces pathogen growth or colonization on or within the polymeric mesh.

In embodiments, the surface functionalization of polymeric mesh material(s) can enhance interaction with biological species within the extracellular matrix (ECM) and/or immobilization of molecules designed to elicit specific biological responses, such as, for example, cell adhesion, attachment, migration, or taxis, through, for example, electrostatic interactions.

In disclosed embodiments, mesh surfaces can be functionalized with, for example, cationic, anionic, zwitterionic, or neutral (non-ionic) functional groups which will interact, or prevent interaction, with specific biomolecules or cells within the ECM. Additionally, in concert or separately, polymeric matrix materials can be modified with reactive surface chemistries which are suitable for covalent interfacial reactions for the permanent immobilization of biologically active molecules. Reactive surface species can comprise amine, carboxy, hydroxy, aldehyde, epoxy, and sulfhydryl groups, and can be grafted to biomolecules using traditional coupling/crosslinking chemistries. There are several different ways to modify surface chemistries that make use of radical, cationic or anionic polymerization. In embodiments, these processes have the net effect of rebuilding the ECM around the functionalized synthetic matrix, and promoting cellular ingress and growth.

### Cationic Functionalization

In disclosed embodiments, functional cationic species can comprise ammonium, guanidinium, phosphonium, pyridinium, and sulfonium groups. Additionally, multivalent metal cations, such as Fe³⁺, Cr³⁺, Al³⁺, Ba²⁺, Sr²⁺, Ca²⁺, and Mg²⁺ and/or polycations, for example polylysine, polyarginine and others, can be used to provide intermolecular attraction.

For example, Mg²⁺ complexed with oxygen groups of anti-adhesive non-ionic polymers provides a synergistic effect by increasing the efficiency of anti-adhesion mechanisms. In general, the cationic groups can charge-couple with the negatively charged polar headgroups of the phospholipid bilayer which is the major component of all cell membranes, thereby attaching the cell.

Alternatively, the cationic species can charge-couple with biomaterials within the ECM which can then interact with cells via their biological responses. For example, at a physiological pH of 7.4, protonation of surface amines will lead to a positive charge that attracts the negatively charged adhesive glycoproteins, such as fibronectin. Fibronectin binds collagen and cell surface integrins, which causes a reorganization of the cell's cytoskeleton and facilitates cellular movement and differentiation. In a like manner, cationic species will charge-couple with proteoglycans, polysaccharides, and collagen which will elicit their biological response under physiological conditions.

Anionic, Zwitterionic, or Neutral (Non-ionic) Functionalization

In disclosed embodiments, mesh surfaces can be charge-modified (i.e. anionic, zwitterionic, or neutral) by grafting various polymers, for example polysaccharides, polypeptoids, polyzwitterions, poly(ethylene glycol) (PEG), polyoxazolines, polyglycerol (PG) dendrons, and glycomimetic polymers. The effects of these polymers/compounds on cell agglutination can involve the blocking of certain cell surface receptors and the activation of others, such as those involved in the attachment to extracellular surfaces and which thereby mediate cellular adherence. For example, it has been demonstrated that cell adhesion in tissues is minimized by employing PEG, which formed a steric barrier between tissues.

Disclosed functional anionic species can comprise carboxylate, phosphate, sulfate and sulfonate groups which increase the polymers hydrophilic nature. This feature, along with electrical neutrality and a hydrogen-bond acceptor/donor chemical structure are common features among many non- or anti-adhesive material classes, such as, PEGs, polyamides, and polysaccharides. Several polysaccharides demonstrate non- or anti-adhesive performance, including heparin, carboxymethylcellulose, dextran, hydroxyacrylates, and hyaluronic acid, any of which can be suitable for use in various embodiments herein.

Studies have shown that a zwitterionic surface having both hydrophobic and lipophobic properties resists protein absorption. Larger microorganisms and proteins are inherently amphiphilic, and can operate by different attachment mechanisms, with some having an affinity to hydrophobic surfaces, and others to hydrophilic. Therefore, solely hydrophilic or hydrophobic surfaces are often inadequate in resisting adhesion formation upon prolonged exposure to complex environments, such as blood. Thus, disclosed embodiments comprise polyzwitterionic species with antifouling properties, for example polybetaines which carry a positive and negative charge on the same monomer unit, such as sulfobetaine methacrylate (SBMA) and carboxybetaine methacrylate (CBMA).

Another class of polyzwitterionic materials suitable for use in disclosed embodiments is the polyampholytes, which carry a 1:1 positive-to-negative charge on two different monomer units, such as natural amino acids. In embodiments, a nanoscale homogenous mixture of balanced charge groups from polyzwitterionic materials is utilized to achieve non-fouling properties. Deviation from charge neutrality can induce electrostatic interactions between proteins and polymer surface, leading to protein adsorption. It is also thought that the polyhydrophilic and polyzwitterionic materials are correlated with a hydration layer near the surface, because a tightly bound water layer forms a physical and energetic barrier to prevent protein absorption on the surface.

Further, functionalized polymer chain flexibility (i.e. surface packing and chain length) plays a role in protein resistance; when protein approaches the mesh surface, the compression of the polymer chains causes steric repulsion to resist protein adsorption due to an unfavorable decrease in entropy. Neutral (non-ionic) polymers also consist of hydrophilic groups (e.g. amides, ethers) which are able to interact with water molecules, as well as hydrophobic groups (e.g. vinyl backbone). PEG for example is a neutral, hydrophilic polyether with hydroxyl end groups which have significant influence on its chemical and physical properties. PEGs have been extensively applied to protein functionalization, for example to extend half-life, and have demonstrated product safety.

Disclosed embodiments can comprise anti-adhesive materials which are either natural (i.e. animal or plant based) polymers, modified natural polymers, or synthetic polymers. For example, disclosed anti-adhesive materials can comprise, alone or in combination, solutions, aerosols, foams, hydrogels, or as solid materials in the form of films or fibers, the antiadhesive/ antifouling polymers chondroitin sulfate, dextran, carboxymethyl dextran, hyaluronic acid, alginate, pectin, cellulose, carboxymethyl cellulose, carboxyethyl cellulose, oxidized regenerated cellulose, chitin, carboxymethyl chitin, carboxymethyl chitosan, polymannuronic acid, polyglucuronic acid, polyguluronic acid, poly(ϵ-caprolactone), polyvinylpyrrolidone, PTFE, expanded PTFE (ePTFE), polyethylene glycol (PEG), PEG stearate, PEG sorbitan monolaurate, polypropylene glycol, polypropylene, polyester, and the like. In disclosed embodiments, the application of PEG produces an anti-adhesive surface in a manner comparable to that as already described for anionic and zwitterionic polymers. For example, in embodiments, PEG is immobilized to a hydrogel-based hernia mesh engineered with a rapid biodegradation profile using traditional coupling chemistry.

As is generally known, excessive hydration is detrimental to bio-adhesion. Therefore, increased supramolecular association can be achieved by carboxylating polymers of the mesh surface. In embodiments, by increasing the density of carboxyl residues on the polymer, it is more likely to hydrogen bond (with water) even at relatively high pH. Therefore, in embodiments, these types of biopolymers can form hydrated gels which act as physical barriers to separate tissues from each other during healing, so that adhesions between adjacent structures do not form.

### Bioactive, Adherent, Bacteriostatic, and Bactericidal Functionalization

Additional mesh layer components for scaffolding support and/or induction of the rate of cellular in-growth, either in combination with or separately from ionic charge functionality, can comprise impregnation or “seeding” with materials. In embodiments, the materials can be natural or synthetic, and can be cross-linked or not by various reagents commonly known in the art.

For example, additional mesh components can comprise, alone or in combination, collagen, gelatin, hyaluronic acid, chitosan, alginate, agar, kappa-carrageenan, heparin, cellulose, starch, PEG, PBLG, polyacrylic acids, polyacrylamides, polyethylene oxide, polyvinyl alcohols, polyvinyl pyrrolidones, fibronectin, vitronectin, tenascin, laminin, chondroitin sulfate, albumin, maltodextrin, elastin, glycosaminoglycans, polyglycans, polypeptides, keratin, organically modified silica, pectins, polyhydroxybutyrates, copolymers of polyesters, polycarbonates, polyanhydrides, polysaccharides, polyhydroxyalkanoates, amino acid residues, and amino acid sequences.

Additional mesh components can comprise, alone or in combination, antimicrobials, such as biguanides or quaternary amines which can be immobilized to the mesh surface via charge and/or crosslinking chemistry. In embodiments, these species can serve a dual role of cellular in-growth activation and anti-microbial protection. These functional groups can be bound to bioactive moieties, such as enzymes, antibodies, proteins, lipids, fatty acids, amino acid residues, and glycosaminoglycans (GAGs) via traditional coupling/crosslinking chemistry.

Further methods for associating additional components to a disclosed surgical mesh can comprise physical rather than chemical bonding. For example, components such as antimicrobials can be physically bound to disclosed mesh scaffolding by solvent casting or “dip coating,” wherein a mesh substrate is immersed (“dipped”) at a constant speed into a solution containing a coating material. After immersion for a fixed period of time, the mesh is removed from the solution at a constant speed. After drainage of excess liquid from the mesh, the solvent evaporates from the liquid, forming a thin layer. For example, silane-anchored antimicrobials can be adhered to porous surfaces such as that of disclosed surgical mesh by dip coating. An advantage of dip coating is that the mesh retains its porosity rather than becoming an impervious film from this process, and this porosity can be of importance in cell in-growth. Further, the use of dip-coating reduces or eliminates the need for pre-treatment of the surgical mesh.

Further disclosed embodiments can comprise biologics such as, for example, gelatin, collagen, HA, growth promoters, TGF-β, FGF, VEGF, combinations thereof, and the like.

### Commercial Products/Kits

The present surgical mesh and associated materials can be finished as a commercial product by the usual steps performed in the present field, for example by appropriate sterilization and packaging steps. For example, the material can be treated by UV/vis irradiation (200-500 nm), for example using photo-initiators with different absorption wavelengths (e.g. Irgacure 184, 2959), preferably water-soluble initiators (e.g. Irgacure 2959). Such irradiation is usually performed for an irradiation time of 1-60 min, but longer irradiation times may be applied, depending on the specific method. The material according to the present disclosure can be finally sterile-wrapped so as to retain sterility until use and packaged (e.g. by the addition of specific product information leaflets) into suitable containers (boxes, etc.).

According to further embodiments, the surgical mesh material can also be provided in kit form combined with other components necessary for administration of the material to the patient. For example, disclosed kits, such as for use in surgery and/or in the treatment of injuries and/or wounds, can further comprise, for example, a hemostatic material and at least one administration device, for example a buffer, a syringe, a tube, a catheter, forceps, scissors, gauze, a sterilizing pad or lotion.

The kits are designed in various forms based on the specific deficiencies they are designed to treat.

### Methods of Use

Methods of use of disclosed embodiments can comprise performing a surgical procedure that utilizes a disclosed surgical mesh, for example a hernia repair procedure.

### EXAMPLES

The following non-limiting Examples are provided for illustrative purposes only to facilitate a more complete understanding of representative embodiments. This example should not be construed to limit any of the embodiments described in the present Specification.

### Example 1

### Testing of Various Embodiments

### Demonstrate an Increased Cellular In-growth for Charged Mesh Materials

Commercially available meshes are modified with both positive and negative functional groups, and with a range of surface charge densities. Several other surface modification strategies are used to impart surface charge to the commercial meshes, including direct photo-polymerization of positively and negatively charged acrylates. Initially these methods involve dipping the mesh into solutions with different concentrations of positively and negatively charged acrylates.

These reactive polymerizable resin-impregnated meshes are then photo or radiation polymerized, and thoroughly rinsed to remove unreacted components.

In addition to the dip methods, deposition methods using plasma are also used. These methods involve exposing the commercial meshes to a plasma containing reactive groups (e.g., allylamine). This produces meshes having different amounts of surface charge densities. In this case, these amine groups will be positively charged under physiological conditions. Independent of the method for surface modification, the charge density will be measured with colorimetric methods. An in-vitro scratch assay (Liang, 2007) will be used to compare cellular in-growth of (1) the charged mesh (test), (2) the uncharged mesh (reference), (3) a biological mesh (reference), and (4) a no-treatment control (negative control). Cellular in-growth is compared by measuring the rate of cell migration and the number of cells in the scratched region.

### Example 2

### Testing of Various Embodiments

### Decreased Cellular Attachment of Functionalized Mesh Material

A commercially available mesh is functionalized with anti-adhesive moieties. This is accomplished in a manner completely analogous to the previous example, though using anti-adhesive strategies instead of charge density. In these cases, the coating contains polyethylene glycol side groups. Alternate strategies involve modification of the dip formulation to crosslink into a hydrogel material, as these are also well known as anti-adhesive surfaces. The modification is followed spectroscopically and the anti-adhesive properties are measured with an in vitro colonization assay (Canute, 2012) which is used to compare cellular colonization of (1) the functionalized mesh (test); (2) the non-functionalized, charged mesh (test, from above); (3) the functionalized, charged mesh (test); (4) the non-functionalized, uncharged mesh (reference); and (5) a biological mesh (reference). Cellular colonization is measured by counting number of cells attached, and by evaluating the production of Type I collagen.

### Example 3

### Testing of Various Embodiments

### Optimizing Synthetic Polymer Mesh and its Manufacturing Process

Conventional photolithography meshes composed of different formulations of photo-crosslinked monomers are produced with different formulations and different geometries, and some include lamination of multiple films together. The resulting films are tested for their mechanical integrity, as well as their biodegradability.

### Example 4

### Testing of Various Embodiments

### Combination of Successful Compositions Into a Single Mesh

The data derived from the above experiments is combined and used to create a synthetic mesh with an anti-adhesive side and an enhanced cellular in-growth side, with appropriate structure to support cellular in-growth, adequate tensile strength, and with programmed biodegradation.

### Example 5

### Testing of Various Embodiments

### Subsequent Planned Experiments

Additional experiments can include the effect of biologics to cellular ingress. For example, gelatin could either be ionically immobilized to the charged mesh materials, or be covalently attached using known crosslinking chemistry, such as glutaraldehyde and genipin. Still other experiments can include antimicrobial efficacy over time, by immobilizing antimicrobial, either by ionic, covalent, or solvent dip coated attachment, to the mesh material.

### Example 6

### Use in Hernia Repair

A disclosed surgical mesh comprising a functionalized charged surface is implanted laparoscopically during hernia repair surgery. The mesh provides increased cellular in-growth while reducing visceral tissue attachment.

In closing, it is to be understood that although aspects of the present Specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to, or alternative configurations of, the disclosed subject matter can be made in accordance with the teachings herein without departing from the spirit of the present Specification. Lastly, the terminology used herein is for the purpose of describing particular embodiments only, and is not intended to limit the scope of the present disclosure, which is defined solely by the claims. Accordingly, embodiments of the present disclosure are not limited to those precisely as shown and described.

Certain embodiments are described herein, comprising the best mode known to the inventor for carrying out the methods and devices described herein. Of course, variations on these described embodiments will become apparent to those of ordinary skill in the art upon reading the foregoing description. Accordingly, this disclosure comprises all modifications and equivalents of the subject matter recited in the claims appended hereto as permitted by applicable law. Moreover, any combination of the above-described embodiments in all possible variations thereof is encompassed by the disclosure unless otherwise indicated herein or otherwise clearly contradicted by context.

Groupings of alternative embodiments, elements, or steps of the present disclosure are not to be construed as limitations. Each group member may be referred to and claimed individually or in any combination with other group members disclosed herein. It is anticipated that one or more members of a group may be comprised in, or deleted from, a group for reasons of convenience and/or patentability. When any such inclusion or deletion occurs, the Specification is deemed to contain the group as modified thus fulfilling the written description of all Markush groups used in the appended claims.

Unless otherwise indicated, all numbers expressing a characteristic, item, quantity, parameter, property, term, and so forth used in the present Specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the Specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the disclosure are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present Specification as if it were individually recited herein.

The terms “a,” “an,” “the” and similar referents used in the context of describing the disclosure (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope otherwise claimed. No language in the present Specification should be construed as indicating any non-claimed element essential to the practice of embodiments disclosed herein.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present disclosure so claimed are inherently or expressly described and enabled herein.

1. A synthetic surgical mesh comprising a chemically functionalized polymeric material.

2. The synthetic surgical mesh of claim 1, wherein said polymeric material comprises at least one of polypropylene (PP), polyethylene terephthalate (PET), expanded polytetrafluoroethylene (ePTFE), polycaprolactone (PCL), poly(L-lactide) (PLL), polyglycolic acid (PGA) and copolymers thereof, poly(lactic-coglycolic acid) (PLGA), poly(glycolide-co-caprolactone), and polyglycolide-co-trimethylene carbonate).

3. The synthetic surgical mesh of claim 1, wherein said mesh is biodegradable.

4. The synthetic surgical mesh of claim 1, wherein the surface of said mesh is functionalized with at least one of cationic, anionic, zwitterionic, or neutral (non-ionic) functional groups.

5. The synthetic surgical mesh of claim 4, wherein said cationic functional group comprises at least one of ammonium, guanidinium, phosphonium, pyridinium, sulfonium, Fe³⁺, Cr³⁺, Al³⁺, Ba²⁺, Sr²⁺, Ca²⁺, Mg²⁺, polycations, polylysine, or polyarginine.

6. The synthetic surgical mesh of claim 4, wherein said zwitterionic functional group comprises at least one of a polybetaine, sulfobetaine methacrylate (SBMA), carboxybetaine methacrylate (CBMA), or a polyampholyte.

7. The synthetic surgical mesh of claim 4, wherein said anionic functional group comprises at least one of a carboxylate, a phosphate, a sulfate, a sulfonate, PEG, polyam ides, or a polysaccharide.

8. The synthetic surgical mesh of claim 1, wherein said mesh is non-woven.

9. A kit for use in performing a surgical procedure, comprising the synthetic surgical mesh of claim 1.

10. A method of performing a surgical procedure comprising implantation of the synthetic surgical mesh of claim 1.

11. The method of claim 10, wherein said surgical procedure comprises a hernia repair procedure.
